(19) United States
(12) Patent Application Publication
(10) Pub. No.: US 2019/0099343 A1
(43) Pub. Date: Apr. 4, 2019

(54) Hair-Washing and Hair-Caring Composition and Preparation Method Thereof

(71) Applicant: Opal Cosmetics (Huizhou) Limited, Huizhou (CN)

(72) Inventor: Junzhong Shu, Huizhou (CN)

(21) Appl. No.: 16/176,048

(22) Filed: Oct. 31, 2018

Related U.S. Application Data
(63) Continuation-in-part of application No. PCT/CN2017/108120, filed on Oct. 27, 2017.

(30) Foreign Application Priority Data
Sep. 29, 2017 (CN) 201710914028.X

(51) Int. Cl. A61K 8/37; A61K 8/46; A61K 8/44; A61K 8/42; A61K 8/89; A61K 8/898; A61K 8/73; A61Q 5/02; A61Q 5/12

(52) U.S. Cl. CPC A61K 8/37; A61K 8/463; A61K 8/466; A61K 8/442; A61Q 5/12; A61K 8/89; A61K 8/898; A61K 8/737; A61Q 5/02; A61K 8/42

(57) ABSTRACT

Provided is a hair-washing and hair-caring composition and preparation method thereof, which relates to the field of daily use chemicals. The hair-washing and hair-caring composition comprises the following in parts by weight: 10˜40 parts of surfactant; 0.1˜5 parts of conditioner; 0.1˜3 parts of suspension stabilizer; and 50˜90 parts of water; where the suspension stabilizer is trihydroxystearin preferably. Trihydroxystearin and other components used in the hair-washing and hair-caring composition form a netlike structure with a weaker bonding force through the hydrogen bond and the molecular force, and such a netlike structure having a weaker binding force provides better stability and can be disconnected along with kneading, allowing the conditioner to fully release to contact the hair and function, avoiding hair twining caused by the conventional macromolecular suspension stabilizer, and reducing the addition amount of the conditioner while maintaining a better washing and caring function, thereby reducing the cost of production.

### CROSS-REFERENCE TO RELATED APPLICATION

This application is a continuation-in-part application of PCT patent application No. PCT/CN2017/108120 filed on Oct. 27, 2017, which claims priority to Chinese patent application No. 201710914028.X, filed with the Chinese Patent Office on Sep. 29, 2017 and entitled “A Hair-washing and Hair-caring Composition and Preparation Method thereof”, the contents of all the above are incorporated herein by reference in their entirety.

### TECHNICAL FIELD

The application relates to the field of daily use chemicals, and in particular to a hair-washing and hair-caring composition and preparation method thereof.

### BACKGROUND ART

Hair-washing liquid, also known as shampoo, is a common daily commodity for cleaning hair in our life. Two-in-one shampoo is a hair-washing liquid that has combined hair-washing effect with hair-caring effect by adding the latter to the former. But in principle, to wash is to wash away greasy dirt on the hair, and to care hair is to give the hair proper grease, for making the hair clean, refreshing, smooth and lustrous, where hair-washing conflicts with hair-caring primarily.

The two-in-one shampoo brings hair-washing and hair-caring which are conflicted in demand, together into a single formulation for convenience in use. However, the two conflicting mechanisms of action result in difficulty in the formulation, and phenomena such as layering and demulsifying will occur in the product if without proper processes, eventually leading to serious quality problems or even accidents. In the prior art, in order to address the issue of the formulation stability of the two-in-one shampoo, a macromolecular suspension stabilizer such as Kaposi, TAB, and the like is used by most manufacturers to suspend grease ingredients in the formulation. However, the netlike structure of the macromolecular suspension stabilizer which causes hair twining during hair-washing reduces significantly the efficacy of the conditioner in the formulation, and also makes foam generation very difficult. Accordingly, it is necessary to add an excessive amount of conditioner and foaming agent, where a bad cycle occurs, resulting in increases in cost inefficiency. Even if the consumer is satisfied in terms of the use effect, the formulation cost would be higher. In addition, the difficulty in processing the macromolecular material also makes the cost of the formulation process increased. This is less competitive in the market accordingly.

### DISCLOSURE OF THE INVENTION

An object of the application comprises providing a hair-washing and hair-caring composition, which avoids using a macromolecular suspension stabilizer of a netlike structure, and which adds hair-washing and hair-caring effects while ensuring the formulation stability.

An object of the application also comprises providing a preparation method of the hair-washing and hair-caring composition, which method is simple to operate, requires less for equipment and process conditions, and is production efficient for the above hair-washing and hair-caring composition and is suitable for industrialized large-scale productions.

The embodiments of the application are implemented as follows.

A hair-washing and hair-caring composition comprises the following in parts by weight:

10˜40 parts of surfactant;

0.1˜5 parts of conditioner;

0.1˜3 parts of suspension stabilizer; and

50˜90 parts of water;

where the suspension stabilizer is trihydroxystearin preferably.

Further, in parts by weight, the hair-washing and hair-carving composition comprises 15˜35 parts of surfactant; 0.5˜3.5 parts of conditioner; 0.3˜3 parts of suspension stabilizer; and 60˜80 parts of water, where the suspension stabilizer is trihydroxystearin preferably.

Further, in parts by weight, the hair-washing and hair-carving composition comprises 20˜30 parts of surfactant; 1˜3 parts of conditioner; 0.5˜2 parts of suspension stabilizer; and 60˜80 parts of water, where the suspension stabilizer is trihydroxystearin preferably.

Further, in parts by weight, the hair-washing and hair-carving composition comprises 25˜30 parts of surfactant; 1˜2 parts of conditioner; 1˜2 parts of suspension stabilizer; and 70˜80 parts of water, where the suspension stabilizer is trihydroxystearin preferably.

Further, the surfactant is at least one selected from the group consisting of an anion surfactant, a zwitterion surfactant, and a nonionic surfactant.

Further, the surfactant comprises a main surfactant and an auxiliary surfactant, and the main surfactant is an anion surfactant, and the auxiliary surfactant preferably comprises at least one of the zwitterion surfactant and the nonionic surfactant.

Further, the main surfactant and the auxiliary surfactant have a mass ratio of any one of 28:9, 18:3.5, 22:4.5, 23:17, 21:8, 16:8, and 16:12.

Further, the anion surfactant is at least one selected from the group consisting of sodium lauryl sulfate, ammonium lauryl sulfate, sodium dodecylbenzene sulfonate, sodium laureth sulfate, ammonium laureth sulfate, and sodium alpha-olefin sulfonate.

Further, the zwitterion surfactant is at least one selected from the group consisting of amino acid surfactant, cocamidopropyl betaine, imidazolinium betaine, dodecyldimethyl betaine, and dodecyldimethylamine oxide.

Further, the nonionic surfactant is at least one selected from the group consisting of polyglucoside, coconut monoethanol amide, and coconut diethanol amide.

Further, the conditioner is at least one selected from the group consisting of polydimethylsiloxane, amino silicone oil, and cyclopentylsiloxane.

Further, in parts by weight, the hair-washing and hair-caring composition further comprises 0.1˜1 parts of antistatic agent.

Further, the antistatic agent is at least one selected from the group consisting of cationic guar, vinylpyrrolidone, acrylamide, and quaternized serine.

Further, in parts by weight, the hair-washing and hair-caring composition further comprises 0.5˜2 parts of pearlescent agent.

Further, the pearlescent agent is at least one selected from the group consisting of ethylene glycol distearate, ethylene glycol monostearate, and pearl pulp.

Further, in parts by weight, the hair-washing and hair-caring composition further comprises 0.1˜2 parts of functional additive, where the functional additive is at least one selected from the group consisting of an anti-dandruff agent, a preservative, a aromatic agent, a buffer, and a colorant.

The application also provides a preparation method of the above hair-washing and hair-caring composition, which comprises mixing a surfactant, a conditioner, a suspension stabilizer, and water.

Further, the preparation method comprises: mixing the surfactant, the suspension stabilizer and the water firstly, heating to 85˜90° C., and stirring to dissolve completely to obtain a mixed solution; and then cooling to below 45° C., blending the mixed solution with the conditioner, and stirring uniformly.

Further, the preparation method comprises: mixing an anion surfactant and a zwitterion surfactant in the surfactant, the suspension stabilizer and the water firstly, heating to 85˜90° C., stirring to dissolve completely to obtain a mixed solution; and then cooling to below 45° C., blending the mixed solution and the conditioner, and stirring uniformly, followed by adding a nonionic surfactant in the surfactant, and stirring uniformly.

Further, the preparation method comprises: before cooling the mixed solution to below 45° C., blending the mixed solution with the antistatic agent after the antistatic agent is dispersed with water below 30° C., and stirring uniformly.

The embodiments of the application include beneficial effects as follows.

The embodiments of the application provide a hair-washing and hair-caring composition comprising a surfactant, a conditioner, a suspension stabilizer and water. Herein, the suspension stabilizer is trihydroxystearin. Trihydroxystearin is used as the suspension stabilizer in the hair-washing and hair-caring composition provided in the application, wherein the hydrophilic groups of trihydroxystearin can form micelles with the surfactant by means of hydrogen bonds, and the lipophilic group of trihydroxystearin and the conditioner are cross-linked mutually and dissolved by the molecular force, forming a netlike structure having a relatively weak binding force, and thus the suspension stability of the hair-washing and hair-caring composition can be maintained better. During the use of the product, such a netlike structure having a relatively weak binding force can be disconnected along with kneading, making the conditioner fully released to contact the hair. Different from the conventional hair-washing and hair-caring products which use covalently-bonded macromolecular suspension stabilizers of a netlike structure, the hair-washing and hair-caring composition provided by the embodiments of the application avoids hair twining caused by the suspension stabilizer, making the conditioner fully contact the hair and take effect, reducing the addition amount of the conditioner while maintaining a better washing and caring effect, thereby reducing the cost of production.

The application further provides a preparation method of the hair-washing and hair-caring composition, which method is simple to operate, requires less for equipment and process conditions, and is production efficient for the above hair-washing and hair-caring composition by simple operations of mixing and is suitable for industrialized large-scale productions.

### DETAILED DESCRIPTION OF EMBODIMENTS

In order to make the object, the technical solutions, and the advantages of the embodiments of the application clearer, the technical solutions in the embodiments of the application will be described clearly and completely. The conventional conditions or conditions recommended by the manufacturer would apply if no specific conditions are indicated in the embodiments. The reagents or instruments used are all conventional products that can be purchased commercially if no manufacturer is indicated.

The hair-washing and hair-caring composition and preparation method thereof of the embodiments of the application will be specifically described below.

A hair-washing and hair-caring composition comprises the following in parts by weight:

10˜40 parts of surfactant; 0.1˜5 parts of conditioner; 0.1˜3 parts of suspension stabilizer; and 50˜90 parts of water, where the suspension stabilizer is trihydroxystearin preferably.

Preferably, in parts by weight, the hair-washing and hair-carving composition comprises 15˜35 parts of surfactant; 0.5˜3.5 parts of conditioner; 0.3˜3 parts of suspension stabilizer; and 60˜80 parts of water, where the suspension stabilizer is trihydroxystearin preferably.

Further preferably, in parts by weight, the hair-washing and hair-carving composition comprises 20˜30 parts of surfactant; 1˜3 parts of conditioner; 0.5˜2 parts of suspension stabilizer; and 60˜80 parts of water, where the suspension stabilizer is trihydroxystearin preferably.

More preferably, in parts by weight, the hair-washing and hair-caring composition comprises 25˜30 parts of surfactant; 1˜2 parts of conditioner; 1˜2 parts of suspension stabilizer; and 70˜80 parts of water, where the suspension stabilizer is trihydroxystearin preferably.

Also referred to as hydroxystearin, trihydroxystearin is a fatty, waxy or powdery solid substance in brown to light brown, and is a mixture of partially oxidized stearic acids and glycerides of other fatty acids, and is soluble in ether, hexane, chloroform and trichloromethane; and it has mild flavor and has a content of 98%. It has the following specific structure:

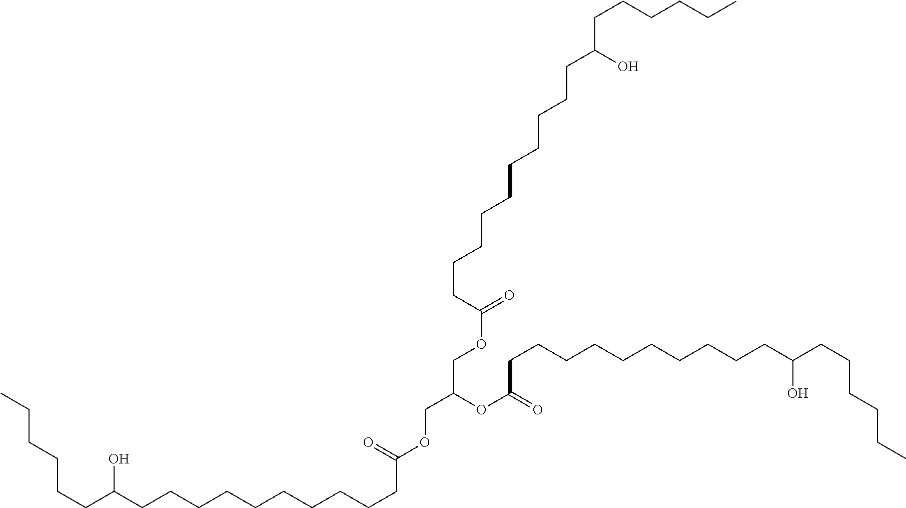

Trihydroxystearin is usually used as an anti-crystallization agent, a chelating agent and a defoamer etc. in vegetable oil, salad oil and cooking oil. Obtained by esterification between stearin and glycerol, trihydroxystearin per se is a lipid substance, having general characteristics of oil and grease, but having weak hydrophilicity due to the hydroxyl group formed after the peroxidation. In general, trihydroxystearin has no water-soluble performance, but in formulations containing surfactants, the hydrophilic group of trihydroxystearin (hydroxyl group) may form micelles with the surfactant by means of hydrogen bonds, while lipophilic groups (aliphatic chains) and non-polar parts (grease and silicone oil, etc.) in the conditioner are cross-linked mutually and dissolved by the molecular force, forming an “octopus” type netlike structure around trihydroxystearin in the formulation solution of the application, thereby maintaining suspension stability of respective ingredients in the formulation.

In the prior art, in two-in-one shampoo, the suspension stability of respective ingredients in the formulation is generally realized dependent on macromolecular polymers (for example, polyacrylic acid, polyoxyethylene, cellulose, etc.) of a netlike structure, and such netlike structure is formed by relatively strong covalent bonds. During hair-washing, these macromolecular polymers of a netlike structure would twine the hair, making it difficult for the conditioner in the shampoo to contact the hair and affecting the generation of foam. Consequently, it is usually necessary to add an excessive amount of conditioner and foaming agent, so as to achieve the desired washing and caring effects. In addition, the difficulty in processing such macromolecular polymer also increases the cost of the formulation and process of the shampoo on the whole. It is less competitive in the market accordingly.

In contrast, in the embodiments of the present application, trihydroxystearin forms a netlike structure with surfactants and conditioners, etc. through weak forces such as hydrogen bond and intermolecular force. Such netlike structure has relatively weak binding, where during hair washing, the netlike structure may be disconnected by gentle kneading, making the conditioner released. Meanwhile, since the netlike structure is disconnected easily, trihydroxystearin would not twine the hair, ensuring full contact of the conditioner with the hair. Only a small amount of conditioners are required to achieve the same washing and caring effect as compared with the prior art, such that the production cost of the two-in-one shampoo is greatly reduced, the market competitiveness of the product is improved, and significant social and economic benefits are obtained.

In the embodiments of the application, the surfactant comprises at least one of an anion surfactant, a zwitterion surfactant, and a nonionic surfactant. Further preferably, the surfactant comprises a main surfactant and an auxiliary surfactant, where the main surfactant is an anion surfactant, and the auxiliary surfactant preferably comprises at least one of the zwitterion surfactant and the nonionic surfactant. Further preferably, the main surfactant and the auxiliary surfactant have a mass ratio of any one of 28:9, 18:3.5, 22:4.5, 23:17, 21:8, 16:8, and 16:12

Herein, the anion surfactant has the functions of foaming and cleaning. It has the advantages of high foaminess, low residual amount, easy formation of micelles and the like, and is usually used as the main surfactant in a hair-washing liquid. Preferably, in the embodiments of the application, the anion surfactant comprises at least one of sodium lauryl sulfate, ammonium lauryl sulfate, sodium dodecylbenzene sulfonate, sodium laureth sulfate, ammonium laureth sulfate, and sodium salt alpha-olefin sulfonate.

The anion surfactant has excellent cleaning power. With strong degreasing power, overuse of the anion surfactant would damage the hair. Therefore, it is necessary to add an auxiliary surfactant to reduce stimulation of the system, adjust the consistency, and stabilize the system. The zwitterion surfactant and the nonionic surfactant are usually auxiliary surfactants. Preferably, in the embodiments of the application, the zwitterion surfactant comprises at least one of an amino acid surfactant, cocamidopropyl betaine, imidazolinium betaine, dodecyldimethyl betaine, and dodecyldimethylamine oxide. The nonionic surfactant comprises at least one of polyglucoside, coconut monoethanol amide, and coconut diethanol amide.

The conditioner preferably comprises at least one of polydimethylsiloxane, amino silicone oil, and cyclopentylsiloxane. The above silicon-containing conditioner can alleviate the unsmoothness of the hair in contact with the hair-washing liquid when combing wet hair, which unsmoothness disappears in the early stage of drying, but presents the next day. Such unsmoothness can be alleviated greatly by adding silicon-containing conditioner which makes the hair smooth, refreshing and bright.

Further preferably, in parts by weight, the hair-washing and hair-caring composition further comprises 0.1˜1 parts of antistatic agent. The antistatic agent comprises at least one of cationic guar, vinylpyrrolidone, acrylamide, and quaternized serine. The antistatic agent, as a cationic compound, has high activity and good compatibility with the anion surfactant, and may exert antistatic effect by depositing on the surface of hair. The cooperation of the antistatic agent and the conditioner can greatly improve the decoration and conditioning of the hair, having clear effects on the conditioning, moisture-maintaining, lustrous, flyaway, smooth and refreshing performances of the hair.

In parts by weight, the hair-washing and hair-caring composition provided by the embodiments of the application further comprises 0.5˜2 parts of pearlescent agent. The pearlescent agent comprises at least one of ethylene glycol distearate, ethylene glycol monostearate and pearl pulp. The pearlescent agent is compatible greatly with other types of surfactants. The pearlescent agent dissolves or emulsifies after being heated in the surfactant, and precipitates lens-like crystals in the cooling process. The pearlescent agent can produce obvious pearlescent effect when used in liquid washing-products, and also has functions such as moistening the skin, nourishing and caring the hair and being antistatic.

Further, in parts by weight, the hair-washing and hair-caring composition provided by the embodiments of the application further comprises 0.1˜2 parts of functional additive. Herein, the functional additive preferably comprises at least one of an anti-dandruff agent, a preservative, a aromatic agent, a buffer, and a colorant. The types of the functional additive are not limited to the above-listed ones and may be appropriately added according to actual needs.

The embodiments of the application also provide a preparation method of the above hair-washing and hair-caring composition, which comprises: mixing a surfactant, a conditioner, a suspension stabilizer, and water, which method is simple to operate, requires less for equipment and process conditions, and is production efficient for the above hair-washing and hair-caring composition and is suitable for industrialized large-scale productions.

Further preferably, the preparation method comprises: mixing the water, the surfactant and the suspension stabilizer and firstly, heating to 85˜90° C., and stirring to dissolve completely to obtain a mixed solution. Then cooling to 10˜45° C., blending the mixed solution with the conditioner, and stirring uniformly. Adding components batchwise based on their own characteristics to allow the uniform mixing of the hair-washing and hair-caring composition.

Specifically, the preparation method of the hair-washing and hair-caring composition comprises the steps as follows.

S1. Firstly adding 75˜85 vol % of water, and then adding the anion surfactant, the zwitterion surfactant and the suspension stabilizer, dispersing uniformly at a high speed, then heating to 85˜90° C. and stirring to dissolve completely.

S2. Dispersing the antistatic agent with an appropriate amount of cold water, adding the dispersed antistatic agent to the resulting solution in S1, keeping it at 80˜85° C., and continuing to stir for 10˜30 minutes. Herein, cold water has preferably a temperature of less than 30° C. Further preferably, cold water has a temperature of less than 20° C.

S3. Cooling to below 45° C., taking 5 vol % of the resulting solution in S2 to mix with the conditioner, homogenizing by a homogenization motor for 10-20 minutes, and dispersing them uniformly, then mixing them with 95 vol % of the the left resulting solution in S2, and stirring uniformly.

S4. Adding the nonionic surfactant to the resulting solution in S3, and stirring uniformly.

S5. Adding other additive and the balance water and stirring uniformly.

A part of the above steps may be omitted and the order of the above steps may be adjusted based on the specific composition of the hair-washing and hair-caring composition.

The feature and the performance of the application are described below in more detail with the embodiment.

### Embodiment 1

The embodiment provides a hair-washing and hair-caring composition, which comprises:

an anion surfactant (20 kg of sodium laureth sulfate, and 8 kg of sodium dodecylbenzene sulfonate), a zwitterion surfactant (2 kg of cocamidopropyl betaine, and 2 kg of amino acid surfactant), a nonionic surfactant (5 kg of coconut diethanol amide), a suspension stabilizer (1 kg of trihydroxystearin), a conditioner (2 kg of polydimethylsiloxane, and 2 kg of amino silicone oil), an antistatic agent (1 kg of cationic guar), a pearlescent agent (2 kg of pearl pulp), and balance water.

Its preparation method comprises steps as follows.

S1. Firstly adding 80 vol % of water, firstly adding the anion surfactant, the zwitterion surfactant and the suspension stabilizer, dispersing uniformly at a high speed, then heating to 90° C., stirring at a medium speed (60 r/min) to dissolve completely.

S2. Dispersing the antistatic agent with an appropriate amount of cold water (10° C.), adding to the resulting solution in S1, keep it at 85° C. and continuing to stir for 15 minutes.

S3. Cooling to 45° C., taking 5 vol % of the resulting solution in S2 to mix with the conditioner, homogenizing by a homogenization motor for 10 minutes, and dispersing them uniformly, then mixing them with 95 vol % of the left resulting solution in S2, and stirring uniformly.

S4. Adding the nonionic surfactant to resulting solution in S3, and stirring uniformly.

S5. Adding pearlescent agent and balance water, and stirring uniformly.

### Embodiment 2

The embodiment provides a hair-washing and hair-caring composition, which comprises:

an anion surfactant (10 kg of sodium laureth sulfate, and 8 kg of sodium dodecylbenzene sulfonate), zwitterion surfactant (0.5 kg of cocamidopropyl betaine, and 2 kg of amino acid surfactant), a nonionic surfactant (1 kg of coconut diethanol amide), a suspension stabilizer (0.1 kg of trihydroxystearin), a conditioner (0.1 kg of polydimethylsiloxane, and 2 kg of amino silicone oil), an antistatic agent (0.1 kg of cationic guar), a pearlescent agent (0.5 kg of pearl pulp), 1 kg of anti-dandruff agents, and balance water.

Please refer to Embodiment 1 for the preparation method, and the preparation method in Embodiment 2 is distinguished from that in Embodiment 1 in that in step 5, the anti-dandruff agent and the pearlescent agent are added together.

### Embodiment 3

The embodiment provides a hair-washing and hair-caring composition, which comprises:

an anion surfactant (20 kg of sodium laureth sulfate, and 2 kg of sodium dodecylbenzene sulfonate), a zwitterion surfactant (1 kg of cocamidopropyl betaine, and 0.5 kg of amino acid surfactant), a nonionic surfactant (3 kg of coconut diethanol amide), a suspension stabilizer (0.5 kg of trihydroxystearin), a conditioner (2 kg of polydimethylsiloxane, and 0.1 kg of amino silicone oil), an antistatic agent (0.5 kg of cationic guar), a pearlescent agent (0.5 kg of pearl pulp), 1 kg of preservative, and balance water.

Please refer to Embodiment 1 for the preparation method, and the preparation method in Embodiment 3 is distinguished from that in Embodiment 1 in that in step 5, the preservative and the pearlescent agent are added together.

### Embodiment 4

The embodiment provides a hair-washing and hair-caring composition, which comprises:

a zwitterion surfactant (2 kg of cocamidopropyl betaine, and 20 kg of amino acid surfactant), a nonionic surfactant (5 kg of coconut diethanol amide, and 8 kg of polyglucoside), a suspension stabilizer (1 kg of trihydroxystearin), a conditioner (2 kg of polydimethylsiloxane, and 2 kg of amino silicone oil), an antistatic agent (1 kg of cationic guar), a pearlescent agent (2 kg of pearl pulp), and balance water.

Please refer to Embodiment 1 for the preparation method thereof.

### Embodiment 5

The embodiment provides a hair-washing and hair-caring composition, which comprises:

a zwitterion surfactant (1 kg of cocamidopropyl betaine, and 10 kg of amino acid surfactant), a nonionic surfactant (3 kg of coconut diethanol amide, and 1 kg of polyglucoside), a suspension stabilizer (0.1 kg of trihydroxystearin), a conditioner (0.5 kg of polydimethylsiloxane, and 0.5 kg of amino silicone oil), an antistatic agent (1 kg of cationic guar), a pearlescent agent (2 kg of pearl pulp), 2 kg of anti-dandruff agent, and balance water.

Please refer to Embodiment 2 for the preparation method thereof.

### Embodiment 6

The embodiment provides a hair-washing and hair-caring composition, which comprises:

an anion surfactant (10 kg of ammonium lauryl sulfate, 8 kg of sodium dodecylbenzene sulfonate, and 5 kg of sodium alpha-olefin sulfonate), a zwitterion surfactant (2 kg of dodecyldimethyl betaine, and 5 kg of Imidazolinium betaine), a nonionic surfactant (5 kg of coconut monoethanol amide, and 5 kg of polyglucoside), a suspension stabilizer (3 kg of trihydroxystearin), a conditioner (2 kg of polydimethylsiloxane, and 2 kg of amino silicone oil), an antistatic agent (1 kg of vinylpyrrolidone), and balance water.

Its preparation method comprises the steps as follows.

S1. First add 70 vol % of water, add the anion surfactant, the zwitterion surfactant, and the suspension stabilizer, disperse uniformly at a high-speed, then heat to 90° C., stir at a medium speed (50 r/min) to dissolve completely.

S2. Disperse the antistatic agent by an appropriate amount of cold water (15° C.), add the dispersed antistatic agent to the resulting solution in S1, keep it at a temperature of 85° C. and continue to stir for 20 minutes.

S3. Cool to a temperature of 30° C., take 10 vol % of the resulting solution in S2 to mix with the conditioner, perform homogenization by a homogenization motor for 20 minutes, and disperse them uniformly, then mix them with the remaining 90 vol % of resulting solution in S2, and stir uniformly.

S4. Add the nonionic surfactant to resulting solution in S3, and stir uniformly.

S5. Add the balance water, and stir uniformly.

### Embodiment 7

The embodiment provides a hair-washing and hair-caring composition, which comprises:

an anion surfactant (5 kg of sodium lauryl sulfate), zwitterion surfactant (3 kg of dodecyldimethylamine oxide), a nonionic surfactant (2 kg of polyglucoside), a suspension stabilizer (2 kg of trihydroxystearin), a conditioner (1 kg of polydimethylsiloxane, and 1 kg of cyclopentylsiloxane), an antistatic agent (1 kg of quaternized serine), and balance water.

Please refer to Embodiment 1 for the preparation method thereof.

### Embodiment 8

The embodiment provides a hair-washing and hair-caring composition, which comprises:

an anion surfactant (8 kg of ammonium laureth sulfate, 8 kg of sodium dodecylbenzene sulfonate, and 5 kg of sodium alpha-olefin sulfonate), a zwitterion surfactant (3 kg of amino acid surfactant, 2 kg of dodecyldimethyl betaine, 3 kg of imidazolinium betaine), a suspension stabilizer (2 kg of trihydroxystearin), a conditioner (2 kg of polydimethylsiloxane, and 2 kg of amino silicone oil), an antistatic agent (0.3 kg of vinylpyrrolidone, acrylamide and 0.3 kg of quaternized serine), and balance water.

Please refer to Embodiment 6 for the preparation method thereof.

### Embodiment 9

The embodiment provides a hair-washing and hair-caring composition, which comprises:

an anion surfactant (6 kg of sodium dodecylbenzene sulfonate, and 10 kg of sodium alpha-olefin sulfonate), a nonionic surfactant (8 kg of coconut monoethanol amide), a suspension stabilizer (3 kg of trihydroxystearin), conditioner (3 kg of cyclopentylsiloxane), an antistatic agent (0.8 kg of acrylamide), 1 kg of anti-dandruff agent, and balance water.

Please refer to Embodiment 6 for the preparation method, and the preparation method in Embodiment 9 is distinguished from that in Embodiment 6 in that in step 5, the anti-dandruff agent is also added.

### Embodiment 10

The embodiment provides a hair-washing and hair-caring composition, which comprises:

a zwitterion surfactant (3 kg of cocamidopropyl betaine, and 3 kg of dodecyldimethylamine oxide), a nonionic surfactant (8 kg of coconut diethanol amide), a suspension stabilizer (1 kg of trihydroxystearin), a conditioner (1 kg of polydimethylsiloxane, 1 kg of amino silicone oil, and 1 kg of cyclopentylsiloxane), an antistatic agent (1 kg of vinylpyrrolidone), a pearlescent agent (1 kg of ethylene glycol monostearate, and 0.5 kg of pearl pulp) and balance water.

Please refer to Embodiment 1 for the preparation method thereof.

### Embodiment 11

The embodiment provides a hair-washing and hair-caring composition, which comprises:

a zwitterion surfactant (6 kg of cocamidopropyl betaine), a nonionic surfactant (6 kg of coconut monoethanol amide), a suspension stabilizer (0.8 kg of trihydroxystearin), a conditioner (1 kg of cyclopentylsiloxane, and 1 kg of amino silicone oil), an antistatic agent (0.4 kg of acrylamide), a pearlescent agent (0.8 kg of ethylene glycol distearate) and balance water.

Please refer to Embodiment 1 for the preparation method thereof.

### Embodiment 12

The embodiment provides a hair-washing and hair-caring composition, which comprises:

an anion surfactant (12 kg of ammonium lauryl sulfate), a zwitterion surfactant (8 kg of cocamidopropyl betaine), a nonionic surfactant (8 kg of imidazolinium betaine), suspension stabilizer (2 kg of trihydroxystearin), a conditioner (2 kg of amino silicone oil), an antistatic agent (1 kg of quaternized serine), a pearlescent agent (2 kg of pearl pulp), 1 kg of aromatic agent and balance water.

Please refer to Embodiment 1 for the preparation method, and the preparation method in Embodiment 12 is distinguished from that in Embodiment 1 in that in step 5, the flavoring agent is also added.

### Embodiment 13

The embodiment provides a hair-washing and hair-caring composition, which comprises:

an anion surfactant (10 kg of ammonium laureth sulfate, and 6 kg of sodium alpha-olefin sulfonate), a zwitterion surfactant (6 kg of dodecyldimethyl betaine, and 6 kg of dodecyldimethylamine oxide), a suspension stabilizer (2 kg of trihydroxystearin), a conditioner (2 kg of amino silicone oil, and 1 kg of cyclopentylsiloxane), an antistatic agent (0.5 kg of vinylpyrrolidone, and 0.5 kg of acrylamide), a pearlescent agent (1.5 kg of pearl pulp), 1 kg of anti-dandruff agent, 1 kg of colorant and balance water.

Please refer to Embodiment 1 for the preparation method, and the preparation method in Embodiment 13 is distinguished from that in Embodiment 1 in that in step 5, the anti-dandruff agent, the colorant and the balance water are added together.

### Experimental Example 1

80 ml of experimental sample was made of the hair-washing and hair-caring composition provided in Embodiment 1. The pH value and the visibility value of the experimental sample were measured using a pH meter and a viscosity meter under a fluorescent lamp illumination and at different temperatures, respectively. The appearance and the fragrance of the experimental sample were then judged by senses. The testing result is shown in Table 1.

TABLE 1
The testing result of the stability of the hair-washing and
hair-caring composition

| | | Observation time and result | | | | | |
|---|---|---|---|---|---|---|---|
| Experiment | Testing | Standard | | | | | |
| condition | item | value | 1 w | 2 w | 4 w | 8 w | 12 w |
| Daylight | appearance | pearlescent | 1/no | 1/no | 2/color | turning | turning |
| lamp | | white | change | change | changing | into | into |
| illumination | | | | | slightly | yellow | yellow |
| | | | | | | slightly | slightly |
| | fragrance | light | 1/normal | 1/normal | 1/normal | 1/normal | 1/normal |
| | | fragrance | | | | | |
| | pH | 5.5-6.5 | 5.88 | 5.85 | 5.75 | 5.88 | 6.05 |
| | viscosity | 13000-15000 cps | 13800 | 14000 | 13600 | 14200 | 13500 |
| | (25° C.) | | | | | | |
| −15° C. | appearance | pearlescent | 1/no | 1/no | 2/color | turning | turning |
| | | white | change | change | changing | into | into |
| | | | | | slightly | yellow | yellow |
| | | | | | | slightly | slightly |
| | fragrance | light | 1/normal | 1/normal | 1/normal | 1/normal | 1/normal |
| | | fragrance | | | | | |
| | pH | 5.5-6.5 | 5.88 | 5.85 | 5.75 | 5.88 | 5.95 |
| | viscosity | 13000-15000 cps | 14200 | 13800 | 14100 | 14000 | 13800 |
| | (25° C.) | | | | | | |
| 5° C. | appearance | pearlescent | 1/no | 1/no | 2/color | turning | turning |
| | | white | change | change | changing | into | into |
| | | | | | slightly | yellow | yellow |
| | | | | | | slightly | slightly |
| | fragrance | light | 1/normal | 1/normal | 1/normal | 1/normal | 1/normal |
| | | fragrance | | | | | |
| | pH | 5.5-6.5 | 5.88 | 5.85 | 5.75 | 5.88 | 6.05 |
| | viscosity | 13000-15000 cps | 14400 | 14000 | 13600 | 14400 | 13500 |
| 25° C. | appearance | pearlescent | 1/no | 1/no | 2/color | turning | turning |
| | | white | change | change | changing | into | into |
| | | | | | slightly | yellow | yellow |
| | | | | | | slightly | slightly |
| | fragrance | light | 1/normal | 1/normal | 1/normal | 1/normal | 1/normal |
| | | fragrance | | | | | |
| | pH | 5.5-6.5 | 5.88 | 5.85 | 5.75 | 5.88 | 6.05 |
| | viscosity | 13000-15000 cps | 13800 | 14000 | 13600 | 14000 | 13800 |
| | (25° C.) | | | | | | |
| 40° C. | appearance | pearlescent | 1/no | 1/no | 2/color | turning | turning |
| | | white | change | change | changing | into | into |
| | | | | | slightly | yellow | yellow |
| | | | | | | slightly | slightly |
| | fragrance | light | 1/normal | 1/normal | 1/normal | 1/normal | 1/normal |
| | | fragrance | | | | | |
| | pH | 5.5-6.5 | 5.88 | 5.85 | 5.75 | 5.88 | 6.05 |
| | viscosity | 13000-15000 cps | 13600 | 13800 | 13500 | 13800 | 13500 |
| | (25° C.) | | | | | | |
| 48° C. | appearance | pearlescent | 1/no | 1/no | 2/color | turning | turning |
| | | white | change | change | changing | into | into |
| | | | | | slightly | yellow | yellow |
| | | | | | | slightly | slightly |
| | fragrance | light | 1/normal | 1/normal | 1/normal | 1/normal | 1/normal |
| | | fragrance | | | | | |
| | pH | 5.5-6.5 | 5.88 | 5.85 | 5.75 | 5.88 | 6.05 |
| | viscosity | 13000-15000 cps | 13300 | 15000 | 13600 | 13600 | 13600 |
| | (25° C.) | | | | | | |
| Three | testing | Initial value | One | Two | Three | — | — |
| cycles | project | | cycle | cycles | cycles | | |
| (48° C.~−15° C.) | | | later | later | later | | |
| | appearance | pearlescent | 1/no | 1/no | 2/color | — | — |
| | | white | change | change | changing | | |
| | | | | | slightly | | |
| | fragrance | light | 1/normal | 1/normal | 1/normal | — | — |
| | | fragrance | | | | | |
| | pH | 5.5-6.5 | 5.88 | 5.85 | 5.75 | — | — |
| | viscosity | 13000-15000 cps | 13900 | 14000 | 13600 | — | — |
| | (25° C.) | | | | | | |

| | | | | | | |
|---|---|---|---|---|---|---|
| level | — | 1 | 1 | 2 | 2 | 2 |

Note:
The testing level comprises levels 1, 2, 3, and 4. Levels 1 and 2 are qualified, and level 3 indicates a conditional pass, that is, the pass needs to be authorized. Level 4 is unqualified.

As can be seen from Table 1, after being subjected to a fluorescent lamp illumination and such conditions as −15° C., 5° C., 25° C., 40° C., and 48° C. for 12 weeks, a hair-washing and hair-caring composition provided by Embodiment 1 of the application still enables to maintain relatively stable pH value and viscosity, resulting in levels 1 and 2 which are qualified under criteria. Meanwhile, the pH value and the visibility were still stable during a course where the temperature changes in a cycle from −15° C. to 48° C., indicating that the hair-washing and hair-caring composition provided by the embodiments of the application has better stability.

### Experimental Example 2

80 ml experimental sample was made of the hair-washing and hair-caring composition provided in Embodiment 3, and the viscosity value was determined using a viscosity meter at different temperatures. The testing result is shown in Table 2.

TABLE 2
Change in the viscosity of the hair-washing and hair-caring
composition with temperature

| | Temperature (° C.) | | | | | | | | | |
|---|---|---|---|---|---|---|---|---|---|---|
| | 5 | 10 | 15 | 20 | 25 | 30 | 35 | 40 | 45 | 50 |
| Viscosity | 16000 | 20000 | 30000 | 32000 | 22400 | 13000 | 8800 | 5200 | 2400 | 1400 |
| (cps) | | | | | | | | | | |

As can be seen from Table 2, at about 20° C., the hair-washing and hair-caring composition provided by Embodiment 2 of the application has the highest viscosity value, and the viscosity value decreased at both a higher temperature and a lower temperature, which shows normal distribution with the temperature point of 20° C. as the center, wherein the contribution temperature for the highest viscosity is close to the environment temperature in daily life. In addition, compared with the hair-washing liquid in the market, the hair-washing and hair-caring composition has a decreased viscosity at a low temperature, with a gentle decreasing slope, avoiding a jelly state formed at a low temperature, which jelly stated affects the consumption experience and use.

### Experimental Example 3

With the hair-washing and hair-caring composition provided in Embodiment 1 and with a commercial product used as a control, the combing force, the hair tensile force and the hair lustrous degree of the hair before and after hair-washing were tested respectively according to HAB/R-WI-125 “Evaluation Method of Hair Cosmetics”, HAB/R-WI-118 “Hair Combing Force Test” and HAB/R-WI-136 “Hair Lustrous Degree Test”, and the testing result is as shown in Table 3.

TABLE 3
Testing result of the hair indicator before and after
hair-washing

| testing content | | Embodiment 1 | Control case |
|---|---|---|---|
| combing force/N | before | 0.0774 | 0.0848 |
| | hair-washing | | |
| | after | 0.0625 | 0.0712 |
| | hair-washing | | |
| | change | −19% | −16% |
| | rate | | |
| Hardness | before | 0.2237 | 0.1497 |
| bending force/N | hair-washing | | |
| | after | 0.1398 | 0.1938 |
| | hair-washing | | |
| | change | −38% | 30% |
| | rate | | |
| lustrous | before | 0.8 | 0.72 |
| degree | hair-washing | | |
| | after | 0.9 | 0.82 |
| | hair-washing | | |
| | change | 13% | 14% |
| | rate | | |

As can be seen from Table 3, with the hair-washing and hair-caring composition provided in Embodiment 1 of the application, the combing force for dried hair after hair-washing is reduced by 19%, indicating that the hair-washing and hair-caring composition has a significant effect of making hair smooth. Meanwhile, with the hair-washing and hair-caring composition, the bending force of the hair after hair-washing is reduced by 38%, indicating that the hair-washing and hair-caring composition has an effect of making the hair soft. Finally, with the hair-washing and hair-caring composition, the lustrous degree of the hair after hair-washing is increased by 13% after the hair is dried, indicating that the hair-washing and hair-caring composition also has a significant effect of making the hair bright and lustrous.

In summary, the embodiments of the application provide a hair-washing and hair-caring composition comprising a surfactant, a conditioner, a suspension stabilizer and water, where the suspension stabilizer is trihydroxystearin. The hydrophilic groups of trihydroxystearin can form micelles with the surfactant by means of the hydrogen bonds, and the lipophilic group of trihydroxystearin and the conditioner are cross-linked mutually and dissolved by the molecular force, forming a netlike structure having a relatively weak binding force, and thus the suspension stability of the hair-washing and hair-caring composition can be maintained better. During the use of the product, such a netlike structure having a relatively weak binding force can be disconnected along with kneading, making the conditioner fully released to contact the hair. Different from the conventional hair-washing and hair-caring products which use covalently-bonded macromolecular suspension stabilizers of netlike structure, the hair-washing and hair-caring composition provided by the embodiments of the application avoids hair twining caused by the suspension stabilizer, allowing the conditioner to fully contact the hair and take effect, reducing the addition amount of the conditioner while maintaining a better washing and caring effect, thereby reducing the cost of production.

The application further provides a preparation method of the hair-washing and hair-caring composition, which method is simple to operate, requires less for equipment and process conditions, and is production efficient for the above hair-washing and hair-caring composition by simple operations of mixing and is suitable for industrialized large-scale productions.

The foregoing is only the preferred embodiments of the application and is not used to limit the application. For those skilled in the art, the application would have various changes and variations. Any modifications, equivalent replacements, improvements, etc. made within the spirit and principles of the application should be covered by the scope of the application.

### INDUSTRIAL APPLICABILITY

The hair-washing and hair-caring composition of the application reduces the addition amount of the conditioner efficiently while maintaining a better washing and caring function, thereby reducing the cost of production. The preparation method of the above hair-washing and hair-caring composition provided by the application has advantages of simple processes and convenient operations, and is suitable for industrialized large-scale productions.

1. A hair-washing and hair-caring composition, comprising, in parts by weight:
10˜40 parts of surfactant;
0.1˜5 parts of conditioner;
0.1˜3 parts of suspension stabilizer; and
50˜90 parts of water,
wherein the suspension stabilizer is trihydroxystearin.

2. The hair-washing and hair-caring composition according to claim 1, comprising, in parts by weight: 15˜35 parts of surfactant; 0.5˜3.5 parts of conditioner; 0.3˜3 parts of suspension stabilizer; and 60˜80 parts of water, wherein the suspension stabilizer is trihydroxystearin.

3. The hair-washing and hair-caring composition according to claim 2, comprising, in parts by weight: 20˜30 parts of surfactant; 1˜3 parts of conditioner; 0.5˜2 parts of suspension stabilizer; and 60˜80 parts of water, wherein the suspension stabilizer is trihydroxystearin.

4. The hair-washing and hair-caring composition according to claim 3, comprising, in parts by weight: 25˜30 parts of surfactant; 1˜2 parts of conditioner; 1˜2 parts of suspension stabilizer; and 70˜80 parts of water, wherein the suspension stabilizer is trihydroxystearin.

5. The hair-washing and hair-caring composition according to claim 4, wherein the surfactant is one selected from the group consisting of an anion surfactant, a zwitterion surfactant, and a nonionic surfactant or any combination thereof.

6. The hair-washing and hair-caring composition according to claim 5, wherein the surfactant comprises a main surfactant and an auxiliary surfactant, and the main surfactant is an anion surfactant, and the auxiliary surfactant comprises at least one of the zwitterion surfactant and the nonionic surfactant.

7. The hair-washing and hair-caring composition according to claim 6, wherein the main surfactant and the auxiliary surfactant have a mass ratio of any one of 28:9, 18:3.5, 22:4.5, 23:17, 21:8, 16:8, and 16:12.

8. The hair-washing and hair-caring composition according to claim 5, wherein the anion surfactant is one selected from the group consisting of sodium lauryl sulfate, ammonium lauryl sulfate, sodium dodecylbenzene sulfonate, sodium laureth sulfate, ammonium laureth sulfate, and sodium alpha-olefin sulfonate or any combination thereof.

9. The hair-washing and hair-caring composition according to claim 5, wherein the zwitterion surfactant is one selected from the group consisting of amino acid surfactant, cocamidopropyl betaine, imidazolinium betaine, dodecyldimethyl betaine, and dodecyldimethylamine oxide or any combination thereof.

10. The hair-washing and hair-caring composition according to claim 5, wherein the nonionic surfactant is one selected from the group consisting of polyglucoside, coconut monoethanol amide, and coconut diethanol amide or any combination thereof.

11. The hair-washing and hair-caring composition according to claim 5, wherein the conditioner is one selected from the group consisting of polydimethylsiloxane, amino silicone oil and cyclopentylsiloxane or any combination thereof.

12. The hair-washing and hair-caring composition according to claim 1, further comprising, in parts by weight, 0.1˜1 parts of antistatic agent.

13. The hair-washing and hair-caring composition according to claim 12, wherein the antistatic agent is one selected from the group consisting of cationic guar, vinylpyrrolidone, acrylamide and quaternized serine or any combination thereof.

14. The hair-washing and hair-caring composition according to claim 1, further comprising, in parts by weight, 0.5˜2 parts of pearlescent agent.

15. The hair-washing and hair-caring composition according to claim 14, wherein the pearlescent agent is at least one selected from the group consisting of ethylene glycol distearate, ethylene glycol monostearate and pearl pulp.

16. The hair-washing and hair-caring composition according to claim 1, further comprising, in parts by weight, 0.1˜2 parts of functional additive, wherein the functional additive is one selected from the group consisting of an anti-dandruff agent, a preservative, a aromatic agent, a buffer, and a colorant or any combination thereof.

17. A preparation method of the hair-washing and hair-caring composition according to claim 1, comprising: mixing the surfactant, the conditioner, the suspension stabilizer and the water.

18. The preparation method of the hair-washing and hair-caring composition according to claim 17, comprising: mixing the surfactant, the suspension stabilizer and the water firstly, heating to 85˜90° C., and stirring to dissolve completely to obtain a mixed solution; and then cooling to below 45° C., blending the mixed solution with the conditioner, and stirring uniformly.

19. The preparation method of the hair-washing and hair-caring composition according to claim 17, comprising: mixing an anion surfactant and a zwitterion surfactant in the surfactant, the suspension stabilizer and the water firstly, heating to 85-90° C., stirring to dissolve completely to obtain a mixed solution; and then cooling to below 45° C., blending the mixed solution with the conditioner, and stirring uniformly, followed by adding a nonionic surfactant in the surfactant, and stirring uniformly.

20. The preparation method of the hair-washing and hair-caring composition according to claim 19, comprising: before cooling the mixed solution to below 45° C., blending the mixed solution with an antistatic agent after the antistatic agent is dispersed with water below 30° C., and stirring uniformly.
